(19) United States
(12) Patent Application Publication
(10) Pub. No.: US 2012/0283193 A1
(43) Pub. Date: Nov. 8, 2012

(54) MANIPULATION OF BRAIN IN A CIRCUIT-SPECIFIC MANNER

(75) Inventors: Nicholas C. Spitzer, Del Mar, CA (US); Davide Dulcis, La Jolla, CA (US)

(73) Assignee: THE REGENTS OF THE UNIVERSITY OF CALIFORNIA, Oakland, CA (US)

(21) Appl. No.: 13/505,271

(22) PCT Filed: Oct. 29, 2010
(86) PCT No.: PCT/US10/54864

§ 371 Date: Jul. 24, 2012

Related U.S. Application Data
(60) Provisional application No. 61/256,976, filed on Oct. 31, 2009.

(51) Int. Cl. C12N 5/0793; A61K 31/4375; A61P 25/30; A61P 25/24; A61N 1/00; C12Q 1/02; A61P 25/16; A61K 31/519; A61K 31/495; A61K 38/17; C12N 13/00; A61P 25/18

(52) U.S. Cl. 514/18.1; 435/375; 435/173.1; 514/279; 435/29; 514/257; 514/255.04; 607/1

(57) ABSTRACT

The present invention relates generally to methods, devices and compositions for treating mental, neurological, and cognitive diseases related to deficiencies in the biosynthesis and/or metabolism of neurotransmitters.

### RELATED APPLICATIONS

This application claims the benefit of priority of Provisional Application Ser. No. 61/256,976, filed Oct. 31, 2009, which is incorporated herein by reference.

### REFERENCE TO FEDERALLY SPONSORED RESEARCH

This invention was made with funds from the Government under Grant No. NS015918 from the National Institutes of Health. The government has certain rights in the invention.

### TECHNICAL FIELD

The disclosure relates generally to methods, devices, and compositions for treating mental, neurological and cognitive diseases related to deficiencies in the biosynthesis and/or metabolism of neurotransmitters.

### BACKGROUND

Neurotransmitters are essential for interneuronal signaling, and the specification of appropriate transmitters in differentiating neurons has been related to intrinsic neuronal identity and to extrinsic signaling proteins. The determination of neuronal phenotypes is a substantial developmental challenge, given the complexity of the nervous system. The classical low-molecular-mass, peptide, gaseous, and growth factor neurotransmitters number 50 or more. The appearance of a particular transmitter in a given class of neurons is a crucial step in differentiation because it enables the neurons to communicate with others with which they make synaptic connections. Expression of an incorrect transmitter could isolate neurons from their normal networks. The absence of synaptic signaling could also reduce trophic support from its postsynaptic partners, leading to neuronal death.

Many illnesses and disorders result from the over- or under-production of neurotransmitters. Examples of such illnesses and disorders include psychiatric illnesses such as schizophrenia, manic-depression, obsessive-compulsive disorder, and addiction. Treatment of these disorders is presently available. The primary existing treatment for manic-depressive illness, for example, is pharmacological, involving the use of drugs that affect the metabolism of neurotransmitters. Some drugs block the uptake of transmitters, thus increasing the amount of a transmitter available to bind to neurotransmitter receptors; other drugs deplete the stores of transmitters in the neurons, decreasing the stores of transmitters that the neurons have available to release. These drugs are relatively selective, but have unwanted side effects. A secondary existing treatment for manic-depressive illness, for example, is electroconvulsive (shock) therapy (ECT). This entails generalized stimulation of the nervous system to produce a seizure, and is performed while the patient is anesthetized. Like the current pharmacological treatment of neurological and psychological disorders, ECT is not a focused treatment and has unwanted side-effects. Furthermore, ECT is used principally to treat the most severe cases of cognitive dysfunction that are refractory to pharmacological therapy. Thus, there is a need for a more selective therapy with fewer side effects for treatment of psychological and neurological disorders.

### SUMMARY

This application provides, among others, a method for modulating the neurotransmitter activity of neurons, allowing for the treatment of various psychological and neurological disorders and permitting the screening of potential candidate neuromodulators useful in the treatment of various psychological and neurological disorders and illnesses. In one embodiment, a method of modulating neurotransmitter activity in a neuron associated with the central nervous system is provided. The method includes contacting the neuron with a stimulatory factor that alters the pattern of signaling and leads to changes in neurotransmitter specification in the neuron. The neuron can be a fully differentiated adult neuron or embryonic neuron. The stimulatory factor can be electrical, light or chemical. The neurotransmitter can be acetylcholine, nitric oxide, histamine, noradrenaline, a bioactive amine, an amino acid or a neuropeptide. Generally, the modulation of neurotransmitter activity comprises altering neurotransmitter expression.

Previously there was no way to replace a neurotransmitter or introduce neurons expressing a specific neurotransmitter in the central nervous system non-invasively and without side effects. Our invention triggers metabolic expression of neurotransmitters in a circuit-specific manner. No drugs, surgery, or embryonic stem cell implantations achieve release of physiological levels of neurotransmitters without side effects. A unique feature of our invention is that this can be accomplished by recruiting and reintegrating pools of neurons that already belong to the malfunctioning circuit. These neurons already contact the appropriate targets but lack the appropriate transmitter until it is induced. What makes the invention additionally novel is the timing and the specificity of the response: the effects are observed rapidly and only the activated circuit is affected.

In another embodiment, a method of treating or inhibiting a psychological disorder in a subject is provided. In one embodiment, the method includes administering to the subject a stimulatory or inhibitory factor that alters the pattern of neuronal activity in a desired neuronal circuit or connection of the brain, thereby resulting in the modification of neurotransmitter specification in the neurons. In one embodiment, the psychological disorder is selected from the group consisting of addiction, substance abuse, autism, dyslexia, obsessive-compulsive disorder, generalized anxiety disorder, post-traumatic stress disorder, panic attacks, social phobia, major depression, bipolar disorder and schizophrenia.

The disclosure provides a method of modulating neurotransmitter specification in a neuron associated with the central nervous system, the method comprising contacting the neuron with a stimulatory factor that alters the pattern of Ca²⁺ spike activity of the neuron. The stimulatory factor can be electrical, olfactory or light.

The disclosure provides a method of treating or inhibiting a psychological disorder in a subject, the method comprising administering to the subject a stimulatory or inhibitory factor that alters the pattern of Ca²⁺ spike activity of neurons, wherein the treatment results in the modification of neuronal specification or neurotransmitter produced by the neurons.

The disclosure also provides a method of altering neurotransmitter expression, the method comprising contacting a neuron comprising a nucleic acid sequence encoding a neurotransmitter, or a nucleic acid sequence encoding an enzyme necessary for the biosynthesis of the neurotransmitter, with a stimulatory factor that alters the pattern of spike activity of the neuron.

The disclosure further provides a method of screening for factors that alter neurotransmitter expression in vivo comprising contacting cultures of neurons prepared from developing embryos, loaded with a calcium indicator, with a test stimulatory or inhibitory factor, wherein the neurons are exposed to different levels of the stimulatory or inhibitory factor and time-lapse imaging is used to assess changes in the firing pattern of calcium spikes produced by the neurons.

The disclosure provides a method of treating neurological, psychological, or psychiatric disorders, the method comprising applying one or more stimulant or inhibitory factors to one or more neurons in such a way that the neurons change their expression or production of specific neurotransmitters.

The disclosure also provides a method of modulating neurotransmitter activity in a neuron associated with the central nervous system, the method comprising contacting the neuron with a stimulatory or inhibitory factor that alters the pattern of Ca²⁺ spike activity of the neuron.

### DESCRIPTION OF THE DRAWINGS

FIG. 1A-D shows dopaminergic VSC neurons regulate skin pigmentation. (A) Diagram illustrating the neuronal circuit controlling this behaviour. Glu, glutamate; MC, melanotrope cells. (B) Raising animals in different light levels changes pigmentation. Left, phenotypes on different backgrounds; right, pigmentation adaptation assayed in the boxed regions to the left after different background or incident illumination. (C) Activity of dopamine (DA) suprachiasmatic melanotrope-inhibiting neurons (SMINs) is necessary for regulation of pigmentation. Left, 2 h white adaptation in a larva treated with 10 nM sulpiride for 30 min compared to control; right, pigmentation at various backgrounds in sulpiride-treated larvae and controls. For b and c, N.6 stage-42 larvae raised in the dark. (D) VSC neuron numbers increase during development of larvae raised in 12 h light/12 h dark on a grey background. Left, transverse section through the stage-42 diencephalon (dotted oval) and VSC (dashed circle); right, appearance of TH neurons during development. N≧6 larvae for each stage. Scale bars: b, 1 mm; c, 200 μm; d, 50 μm. Error bars, s.e.m.; **P<0.001.

FIG. 2A-E shows dopaminergic differentiation is activity-dependent. (A) Molecular markers identify VSC neurons (dashed circles) in transverse sections in a control larva and in larvae after sodium (rNav2aab) or potassium (hKir2.1) channel overexpression (stage 42). The VSC is shown at higher magnification (insets). (B) Altering spike activity drives proportional changes in the number of TH neurons. Shown is quantification of data in a. N>6 larvae. (C) Neurons generate calcium spikes in the developing brain. Left, transverse section (dashed line) through the hypothalamus (stage 35) and Fluo-4AM-loaded tissue (below); confocal imaging and staining are from the inset. Right, digitized fluorescence (F) of representative cells circled in the panel below; traces show calcium spike activity recorded from dorsal (top trace, red circle) TH2 and ventral (bottom trace, yellow circle) TH1 cells. N53 larvae. (D) Across developmental stages the number of TH neurons is inversely proportional to the incidence of spiking in LIM1/2 neurons in controls and after alterations of activity. (E) TH mRNA expression is altered by ion channel overexpression. VSC double labelling with TH antibodies and TH-antisense probe (TH-as) in transverse brain sections (top row) and enlargements of the VSC (dashed circles, bottom row) in larvae injected with cascade blue (left panel, control), overexpressing sodium channels (second panel) or potassium channels (third panel). Antisense-probe-binding in sodium-channel-overexpressing larvae (TH-s, fourth panel). Quantification is shown to the right. (A)-(E), Larvae were raised on a 12 h/12 h day/night cycle on a grey background. N≧6 larvae. Scale bars: a, 50 μm; c, 500 μm (left), 30 μm (right); e, 100 μm. Error bars, s.e.m.; **P<0.001.

FIG. 3A-F shows illumination changes the number of dopaminergic neurons selectively in the VSC. (A) Raising animals in different light levels changes the number of VSC TH neurons. N$9 stage-42 larvae. (B), Whole mounts (top) and transverse sections (bottom) show the TH core (dashed inner circle) and annular neurons (arrows, between dashed circles) in 2-h-black- and white adapted stage-42 larvae. Dashed line indicates section orientation. (C) Two hour exposure to different background illumination (B, black; G, grey; W, white) changes the number of TH/NPY neurons in the VSC (core plus annulus). (D) Core and newly TH annular neurons from 2-h-white-adapted larvae are from the LIM1/2 pool; sections as in (B). (E) Exposure to different backgrounds for 2 h changes the number of LIM1/2 neurons expressing TH. (F) TH mRNA expression is altered by light or dark adaptation. Triple-labeling of transverse VSC sections (dashed circles) with TH, NPY and TH-antisense probe in 2-h-dark/black-adapted and 2-h-light-/white-adapted larvae. Merged images (top row) and separate channels (bottom row). Quantification is presented to the right. (B)-(F), Stage-42 larvae raised in the dark. (C), (E), (F) N≧6 larvae. Scale bars: (B) 100 μm (top), 60 μm (bottom); (D) 100 μm; (F) 60 μm. Error bars, s.e.m.; **P<0.001.

FIG. 4A-C shows blocking of physiological activity eliminates illumination dependent changes in the number of dopaminergic VSC neurons. (A) Binocular eye enucleation abolishes white-adaptation- and background illumination-dependent changes in numbers of TH neurons. (B) Implanted beads delivering activity blockers prevent appearance of TH/NPY neurons in larvae exposed to 2 h light relative to untreated or diffusion marker controls. Transverse sections through the VSC (dashed circles) show NPY/TH colocalization (arrows) in white-adapted control (WA control) and calcein-bead-implanted larvae that is absent in TTX and BAPTA-bead-implanted larvae. Scale bar: 40 μm. (C) TH and NPY identify VSC neurons in the WA control and black-adapted (BA) control and after suppression of activity. a, c, N≧6 stage-42 larvae raised in the dark. Error bars, s.e.m.; **P<0.001.

FIG. 5A-F shows NPY neurons projecting to melanotrope cells express TH after illumination. (A) Left, TH is absent in NPY annular neurons (arrows) in transverse sections of 2-h-dark-adapted larvae. Right, TH is present in somata (arrows) and axons (arrowheads) of NPY annular neurons in 2-h-white- and light-adapted larvae; far right, colour separation of green TH and red NPY (insets). (B) Left, TH nerve terminals invest POMC stained melanotrope cells in 2-h-dark-adapted larvae; TH-negative NPY terminals project to POMC-negative melanotrope cells (arrows). Right, TH/NPY terminals project to POMC melanotrope cells (arrows) in 2-h-white adapted larvae. (C) Quantification of (B). (D) D2 receptors are expressed in melanotrope cells of 2-h-light-adapted larvae that newly express or may be acquiring POMC (light grey and black bars). (E) DRAQ5-labelled nuclei identify 2-h-dark/light-dependent changes in melanotrope POMC expression. (F) Quantification of (E). (A-F), Larvae raised in the dark. (C), (D), (F), N≧6 stage-42 larvae. Scale bars: (A) 100 μm; (B, E) 40 μm. Error bars, s.e.m.; **P<0.001.

FIG. 6A-C show newly promoted dopaminergic neurons regulate pigmentation. (A) MPTP selectively eliminates core VSC neurons. Top, TH/NPY in transverse sections before (left) and after (right) 8 h incubation of larvae in 700 μM MPTP in the dark. Middle, the effect of different MPTP concentrations on numbers of TH/NPY VSC neurons. Bottom, 700 μM MPTP eliminates VSC but not dorsolateral suprachiasmatic nucleus (DLSC) and posterior tuberculum (PT) DA nuclei. (B) Top, after MPTP treatment TH is largely absent from NPY annular neurons after dark adaptation (left), and is abundant after 2 h light adaptation (right). Middle, scheme illustrating transmitter expression in annular neurons in these conditions. Bottom, quantification of induction of TH; a, b, N.6 stage-42 larvae raised in the dark. Scale bars: 50 μm. (C) Illumination- dependent changes in skin pigmentation are rescued when MPTP treatment is followed by 2 h exposure to light. Pigmentation of three groups of larvae raised in the dark. Pigmentation was measured before (black bars) or after (white bars) 30 min of white adaptation (WA), or after WA in the presence of sulpiride (grey bars). MPTP-dark, MPTP followed by 2 h incubation in the dark abolishes the change in pigmentation in response to 30 min white-background adaptation. MPTP-light, MPTP followed by 2 h incubation in the light recovers most of the change in pigmentation in response to a 30-min exposure to white background, relative to control (three right bars). Sulpiride (10 nM) during 30-min exposure to white background blocks the reduction in pigmented area (grey bars, centre and right groups). N10 stage-42 larvae. Error bars, s.e.m. **P<0.001.

FIG. 7 shows long daylight exposure decreases the number of tyrosine hydroxylase immunoreactive neurons in the PaVN of the hypothalamus of adult rats. Left, PaVN of an experimental rat; boxed region is enlarged at bottom. Right, PaVN of a control rat; boxed region is enlarged at bottom. Sections were counterstained with Giemsa to reveal cell nuclei.

FIG. 8 shows long daylight exposure reduces by 50% the number of TH-IR interneurons in hypothalamic nuclei of adult rats. PaVN, paraventricular nucleus; LPO, lateral preoptic area. n=3. **, p<0.001.

FIG. 9 shows the effect of one week of long daylight exposure reduces by up to 90% the number of TH-IR interneurons in three hypothalamic nuclei of adult rats. PeVN, periventricular nucleus; PaVN, paraventricular nucleus; LPO, lateral preoptic area. n=3. **, p<0.001.

### DETAILED DESCRIPTION

As used herein and in the appended claims, the singular forms “a,” “and,” and “the” include plural referents unless the context clearly dictates otherwise. Thus, for example, reference to “a cell” includes a plurality of such cells and reference to “the agent” includes reference to one or more agents known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure.

Appropriate expression of neurotransmitters and their receptors is essential for the normal function of the adult nervous system. Abnormal expression causes profound disorders of mental health. The disclosure demonstrates that altering the activation of a neural circuit in the postembryonic brain by natural stimuli (e.g., ambient light, odors and the like) leads to respecification of the neurons, e.g., by changing the number of neurons expressing a particular neurotransmitter or the amount of neurotransmitter released. Promoting the development or respecification of neurons can have an effect on various disease and disorders of the brain including neurological and psychiatric disorders as well as promote development following neuronal injury.

The disclosure indicates that physiological levels of environmental illumination, odor or other natural stimuli dynamically regulate the specification and/or number of neurons in a neuronal circuit. For example, the disclosure demonstrates that physiological levels of environmental illumination or other visual stimuli dynamically regulate the number of dopaminergic VSC neurons innervating hypothalamic melanotrope cells that control pigmentation. Similarly, modulation of olfactory circuits can be achieved using olfactory stimuli (e.g., odors) to cause respecification of neurotransmitters in neurons in the activated neuronal circuit.

In the methods of the disclosure various stimuli can be used to respecify a neuron's neurotransmitters. The stimuli may be light, odor, or other sensory modalities as well as electrical stimulation and the like provided to a particular neuron or circuit. In some embodiment, the stimuli are provided in pulses, concentration or combinations that are not normally found in the environment (e.g., rapid light pulses, repeated frequent olfactory stimulation and the like). Although the stimuli may be similar to natural stimuli the dose, frequency, and the like are provided in a manner that is not natural.

Appropriate postsynaptic receptors are regulated in parallel, as observed during transmitter respecification in the peripheral nervous system. The recruitment of additional neurons is activity- and calcium-dependent and derived from a subpopulation of neurons that display a characteristic molecular signature and already project to a relevant target. For example, when the retinohypothalamic projection is selectively activated, DA synthesis and transport machinery is acquired by annular NPY neurons that surround the TH core of the VSC nucleus.

As previously observed in GABA- and TH-expressing neurons of tottering mutant mice, respecification of the TH phenotype occurs without influencing expression of the default original transmitter (NPY) in the annular neurons, and follows a homeostatic rule. The number of inhibitory dopaminergic neurons increases after enhanced circuit activation due to higher light exposure, increasing inhibitory input to melanotrope cells and decreasing pigmentation.

Conversely, dark-adapted animals respond to lower light-induced activation of this circuit by seducing the number of DA neurons; decreased inhibitory input to the melanotrope cells then boosts pigmentation. This form of plasticity evoked by sensory stimulation parallels use-dependent changes in neuropeptide expression in the hypothalamus and activity-dependent alterations in cortical receptive fields in response to alteration of sensory experience.

The disclosure demonstrates that neurons innervating a class of target neurons express different transmitters but belong to a constellation of cells in which the transmitter phenotype can be expanded (by co-expression) or reduced (by elimination), depending on the physiological requirements of the regulated behaviour. The results of the disclosure indicate a general role of activity in homeostatic specification of monoamines expressed by CNS neurons. The results demonstrate that ectopic neurotransmitter expression can be functionally significant when activity is manipulated by activation of the neural circuit controlling a specific behaviour.

The disclosure demonstrates that physiological stimuli can respecify neurotransmitter expression and functional output of a specific neuronal network by harnessing circuit activity; the nervous system then identifies the right molecules to express, and delivers them at the right time, to the right place and in the right dose. Signaling molecules potentially implicated in this form of plasticity include a-amino-3-hydroxy-5-methyl-4-isoxazolepropionic acid (AMPA)/kainate ionotropic glutamate receptors involved in the light-dependent excitatory response of DA suprachiasmatic neurons and brain derived growth factor, fibroblast growth factor and microRNAs that have roles in establishing the proper number of DA neurons and the like. This approach provides direction for new stimulation methods tuned to activate selected neuronal circuits. Target drug activation and delivery and can prevent or slow the progression of cognitive and neurodegenerative disorders before discovery of the activity-dependent molecular mechanisms involved.

The disclosure describes the use of electrical, optical, olfactory stimuli and the like to activate neuronal projections and circuits. For example, the disclosure describes the use of optical stimuli to modulate the activity via retino-hypothalamic projection to promote transmitter respecification in the adult mammalian brain. Because the retino-hypothalamic-pituitary circuitry is conserved in rodents and in primates, light-induced changes in transmitter specification occur in rodents and humans as well. The methods of the disclosure provide non-invasive therapies for treating mental diseases and disorders directly or as part of a pharmacological treatment.

Electrical and/or chemical neuromodulating techniques are provided. In one embodiment, the disclosure relates to modulation of neuronal activity to affect neurological, psychological, or psychiatric activity. In one embodiment, the disclosure finds application in the modulation of neuronal function or processing to affect a functional outcome. The modulation of neuronal function is useful with regard to the prevention, treatment, or amelioration of neurological, psychiatric, psychological, conscious state, behavioral, mood, and thought activity (unless otherwise indicated these will be collectively referred to herein as “psychological activity” or “psychiatric activity”). When referring to a pathological or undesirable conditions associated with the activity, reference may be made to “psychiatric disorder” or “psychological disorder” instead of psychiatric or psychological activity. Although the activity to be modulated usually manifests itself in the form of a disorder such as addiction, substance abuse, autism, dyslexia, obsessive-compulsive disorder, generalized anxiety disorder, post-traumatic stress disorder, panic attacks, social phobia, major depression, bipolar disorder, or schizophrenia, it is to be appreciated that the invention may also find application in conjunction with enhancing or diminishing any neurological or psychiatric function, not just an abnormality or disorder. Psychiatric activity that may be modulated can include, but not be limited to, normal functions such as alertness, conscious state, drive, fear, anger, anxiety, euphoria, sadness, and the “fight or flight” response. Neurological disorders that may be modulated can include movement disorders such as Parkinson's disease, tardive dyskinesia, and Huntington's disease.

In yet another method the disclosure is useful for respecification of a neuronal circuit that may be damanged from trauma or disease. In such embodiments, the disclosure relates to providing repeated stimuli to a nerve or neuronal circuit to compensate or respecify the circuit to overcome or modify the damaged circuit.

In one embodiment, the disclosure provides methods and compositions for manipulating the electrical activity of the nervous system. The disclosed methods and compositions can be used to modify the identity of the neurotransmitter molecules that nerve cells synthesize and use to communicate with other nerve cells in the central nervous system. For example, the use of Transcranial Magnetic Stimulation (TMS) for modulating the activity and/or synthesis of neurotransmitters is encompassed by the disclosure. TMS applied at frequencies mimicking natural patterns of activity occurring in the brain; for example, frequencies on the order of ten magnetic pulses per hour can be used. Understanding the ways in which neurons express particular transmitters could have a profound impact on the way we think about treating mental illness, many forms of which result from disorders of neurotransmitter metabolism.

In one embodiment, the disclosure provides a more selective and specific therapy for manic-depressive illness, schizophrenia, and perhaps other neurological or cognitive disorders than the pharmacological therapy and the gross electrical stimulation (e.g., electroconvulsive) therapy that presently exist.

In one embodiment, the disclosure relates generally to modulating the pathological electrical and chemical activity of the brain by electrical, light or chemical stimulation and/or direct placement of neuromodulating chemicals within the central nervous system (CNS). For example, drugs that would potentiate activity-dependent, circuit-specific neurotransmitter respecification can be used. Circuit activation would provide the site-specific activation of the drugs, amplify activity at the activated synapses that would lead to changes in transmitter expression in the postsynaptic neurons and repurposed neurons that are wired into circuits. In this embodiment, a caged glutamate compound, for example, in the synaptic cleft from which glutamate is uncaged by the action of synaptically released glutamate can be used. This could also work for BDNF or GABA or other agents. Using such a method the drug does not have to be delivered to a specific location in the CNS, concentration would be low and specific side effects could be minimal.

In yet another embodiment, the disclosure provides for the treatment of, for example, psychiatric disorders (e.g. addictions, substance abuse, obsessive-compulsive disorder, generalized anxiety disorder, post-traumatic stress disorder, panic attacks, social phobia, major depression, bipolar disorder, and schizophrenia). In this embodiment, the disclosure uses neuronal stimulus to modulate calcium flux and/or neurotransmitter production and release in a circuit associated with the disease or disorder. One advantage of the methods of the disclosure is the ability to identify and modulate neuronal circuits at various levels (e.g., expanded circuits comprising 10-100 neurons or specific circuits of 2-10 neurons).

In another embodiment, the disclosure includes an approach to treat mental and related cognitive diseases and movement and related neurological disorders that arise from deficiencies in the biosynthesis/metabolism of certain important neurotransmitters. The approach can be used in combination with a medical device or pharmaceuticals.

In one embodiment, the methods of the disclosure comprise applying electro-, light, odor or other physiological stimulus for a sufficient period to alter production of a specific neurotransmitter, the amount of a specific neurotransmitter or the synaptic interaction of a neuron with a downstream neuron. In this manner the “electrical activity” of the brain or neuronal circuit is modulated (e.g., activity is increased or decreased). It has been discovered herein that repeated stimuli comprising light, electrical or other physiological stimulation of the nervous system or circuit results in the change of identities or production of the neurotransmitters by neurons in the circuit. Therefore, for example, any disease that produces an aberrant amount of specific neurotransmitter (e.g., over production or under production) can be modulated by manipulating its electrical activity. In this way, a different type or different amount of neurotransmitters may be synthesized to compensate the system.

The electrical, olfactory or light stimulation of different regions/circuits of the young or adult brain or regenerating spinal cord can thus lead to a change in the neurotransmitters that neurons synthesize and use to communicate with other neurons. Using these techniques one can modulate neurotransmitter production in subjects known to have a particular disease or disorder associated with over or under production of a particular neurotransmitter or in the mis-production of a neurotransmitter in a particular circuit. Accordingly, the methods of the disclosure provide a treatment for mental disorders, such as depression and schizophrenia, or neurological disorders, such as Parkinson's disease, tardive dyskinesia, and Huntington's disease, that are known to result from disorders of neurotransmitter metabolism. Such treatment would be focused and would avoid the side-effects caused by the existing, more generalized, therapies.

In one embodiment, the disclosure provides a more selective and specific therapy for manic-depressive illness, schizophrenia, and other neurological or cognitive disorders compared to the standard pharmacological therapy and the gross electrical stimulation (e.g., electroconvulsive) therapy that presently exist. In one embodiment, the disclosure identifies a neuron or neuronal circuit to be modified, providing an optical, electrical, or chemical stimulus to the neuron or neuronal circuit for period of time to modify the calcium flux and/or the production of neurotransmitter in the neuron or neuronal circuit, wherein the production of the neurotransmitter is changed either by type or amount.

A neuronal circuit, location or neuron can be identified using methods known in the art. The disclosure can be used in conjunction with magnetoencephalography (MEG). MEG is the measurement of magnetic fields generated by electric currents in the brain. Measurement of these fields close to the surface of the head allows localization of the origin of the electric currents and may be used to map cortical brain function. MEG provides millisecond temporal resolution and millimeter spatial resolution of brain function, but no detailed anatomical information. It is therefore often combined with MR imaging, the merged data set being named magnetic source imaging MSI.

MEG is based on the principle that all electric currents generate magnetic fields. The main source of the extracranial magnetic fields that are detected with MEG instruments is current flow in the long apical dendrites of the cortical pyramidal cells. A distal excitatory synapse will induce a dipolar dendritic current towards the soma of the pyramidal cell, meaning that the electricity is flowing in one direction along the entire length of the dendrite, which therefore may be considered an electric dipole. Pyramidal neurons constitute nearly 70% of neocortical neurons, and the cells are oriented with their long apical dendrites perpendicular to the brain cortex. There are more than 100,000 of these cells per square millimeter of cortex. Dipolar currents flowing in these dendrites induce time-varying magnetic fields perpendicular to the dendrite direction. The pattern of these external magnetic fields can be used to determine the location, orientation and strength of the source electric dipoles.

Neuromagnetic fields have amplitudes in the order of a few picotesla (10-12 tesla) and very sensitive instruments are needed to detect these extremely weak fields. Most MEG instruments (magnetometers, biomagnetometers) are placed in special magnetically shielded rooms. The walls have one or more layers of mu metal, an alloy with very high magnetic permeability, mounted on an aluminum plate serving as magnetic and electromagnetic shielding. External magnetic fields follow the mu metal around the room, away from the interior MEG instrument. MEG detection systems are made from superconductive material immersed in liquid helium. MEG detectors are specially designed coils, the most common one being named axial first-order gradiometer. This coil consists of two coil loops wound in opposite directions, typically less than 4 cm apart. The time-varying external neuromagnetic fields induce electric currents in both loops, the strength of the currents being determined by the strength of the magnetic fields. If the loop currents had identical strengths, they would cancel and no signal would emanate from the detector. Dipolar magnetic fields diminish with the square of the distance from the dipolar source and the loop closest to the brain will therefore experience a slightly stronger field than the loop more distant from the brain. The net output from the detection coil is thus proportional to the magnetic field gradient. The gradient is steep close to the source and shallow far from the source. This makes the detector more sensitive to a very close weak source (such as the brain a few centimeters away), than to a strong, very distant source (such as an MR scanner some hundred feet away).

The detection coil is inductively coupled to a SQUID (superconducting quantum interference device). This is a ring of superconducting material interrupted by two microscopically thin resistive segments (Josephson junctions). A small current is applied to the ring, and provided the current is below a certain critical value, the current will flow without resistance despite the two tiny resistive segments. Any increase in the SQUID current above the critical value will cause a significant drop in the current due to sudden energy loss in the resistive segments. The SQUID current is kept just below the critical value and any induced additional current caused by a net output from the detection coil, will cause a significant drop in the SQUID voltage. The voltage drop is detected by the electronics, which applies a feedback current to counterbalance the induced current in the SQUID. The output from the MEG instrument is determined by the magnitude of the feedback current as measured by a voltmeter. Modern MEG instruments have multiple (e.g., 37) detectors, so-called large-array biomagnetometers. Whole-head systems may have dual multi-channel detectors for simultaneous bilateral recordings, or the dewar containing the multiple detectors may have a helmet-like shape. Large, flat detector systems intended for measurement of biomagnetic fields from the heart, also exist.

The recorded biomagnetic signals are very similar to EEG, and also similar to EEG the signals may be either spontaneous or related to some stimulus (audiovisual, tactile, vibratory, electric, etc.). MEG may be used to explore normal brain function, to map brain function in the vicinity of a tumor or epileptic focus prior to surgery or radiation therapy, to image epileptic foci, to monitor recovery after stroke or head trauma and to study the effects of neuropharmacological agents.

### EXAMPLES

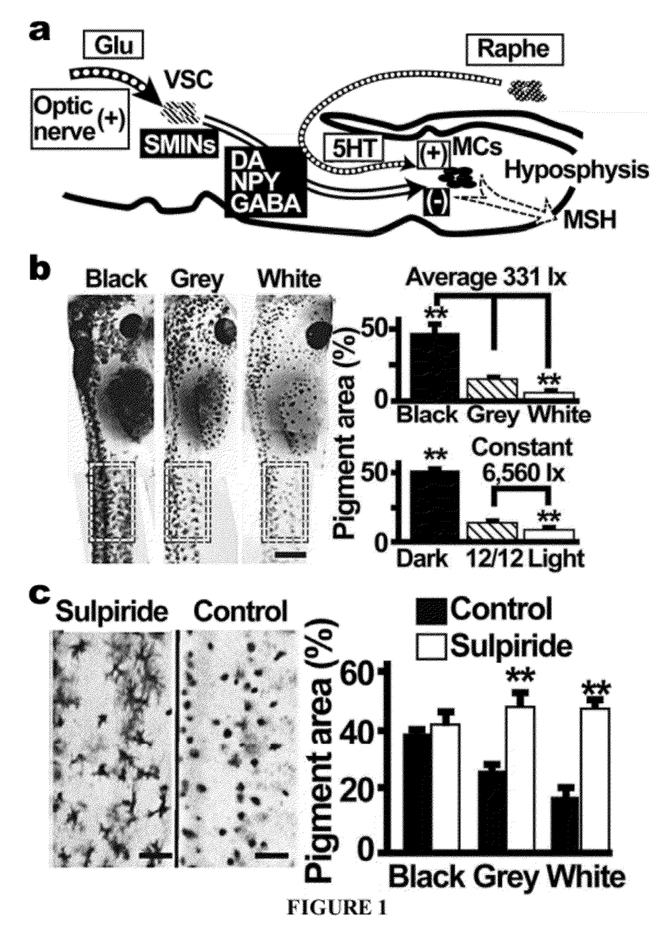

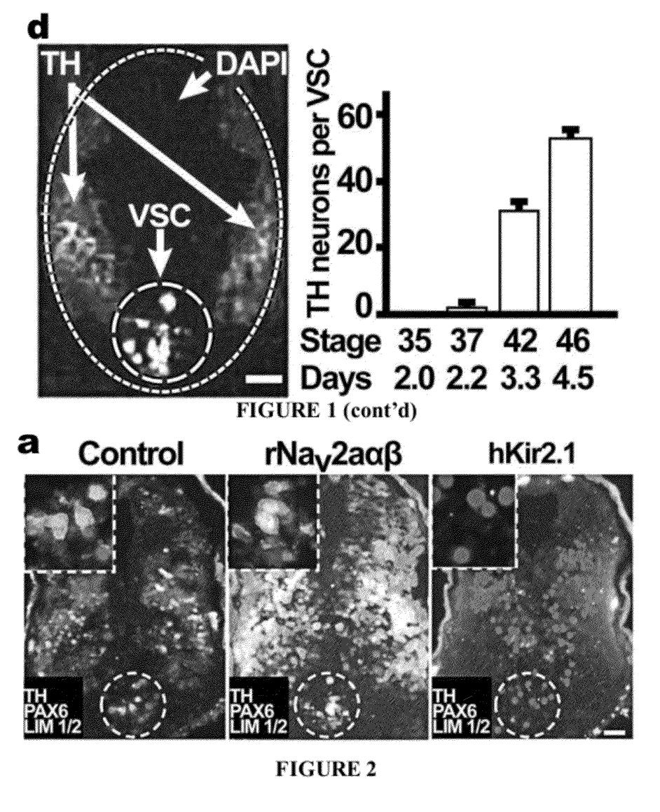

The examples demonstrate methods of the disclosure using olfactory, light and electrical stimuli. The regulation of pigmentation is a well-conserved. behaviour among vertebrate species. Mammals undergo gradual changes in skin colour under hormonal control; many other vertebrates including teleosts and amphibians display rapid physiological colour changes in response to the same hermones. This behaviour mediated by simple neuronal circuitry in Amphibia: glutamatergic retinal ganglion cells project to dopaminergic suprachiasmatic melanotrope inhibitory neurons (SMINs), which innervate cholinergic melanocyte-stimulating-hormone (MSH)-releasing cells (melanotrope cells, FIG. 1a). Epithelial pigmentation of amphibian larvae is regulated by the level of ambient illumination, either by incident light or by background light level, which leads to aggregation and dispersion of melanin granules within melanocytes as in teleosts.

Sustained increases in ambient illumination are expected to increase uptake of 18 F dopa in axon terminals of dopaminergic neurons of the suprachiasmatic nucleus and other TH⁺ hypothalamic interneurons. Binding of ¹¹C raclopride to D2 receptors of hypothalamic targets (SON and PVN) is expected to demonstrate upregulation of receptors.

Illumination experiments. The optimal condition for altering skin pigmentation and numbers of TH neurons were determined by raising larvae under two illumination conditions. In the first, larvae were placed on black, grey or white backgrounds (corresponding to N2.25, N7.25 or N9.5 on the Munsell neutral value scale of a monochromatic colour wheel) with constant illumination (331 lux (lx)). In the second, larvae on a white, grey or black background were exposed to constant illumination (6,560 lx, measured with a light meter (Sper Scientific)-equivalent to shady illumination on a sunny day), or to constant dark or to 12 h light/12 h dark. Light on white background proved more effective than light on a dark background. Subsequently, when testing the effect of light on pigmentation or the numbers of TH neurons, animals were usually raised in the dark (stages 35-42) and exposed (typically for 2 h at stage 42) to these optimal conditions. For some tests of the functional significance of newly TH neurons, animals were raised under different light/background conditions and a conditioning step of 30 min light/white background exposure was imposed to achieve similar levels of skin pigmentation before comparing light or dark adaptation between these groups.

Neuropharmacology. In vivo drug application was achieved with agarose beads, implanted 100-200 μm into the brain of stage-41 larvae. For selective ablation of VSCDA neurons, MPTP was bath-applied in the dark to larvae for 8 h at stage 41. 100 mM deprenyl (Sigma) was bath-applied to inhibit monoamine oxidase.

Immunocytochemistry. The number of neurons in the VSC was quantified by counting the cells within the core and the surrounding annular region in sections of the diencephalon.

In situ hybridization. TH mRNA was detected by locked nucleic-acid-based in situ hybridization optimized for cryostat sections with the tyramide-fluorescein amplification system (Perkin Elmer).

In PET scanning experiments animals analyzed following 5 days of 12:12 hr light:dark exposure and rescanned both after 5 days of constant dark and after 5 days of constant light are expected to exhibit changes in dopamine metabolism that track their environmental exposure.

Pigmentation appears lighter with bright illumination or a white background and darker with dim illumination or a black background (FIG. 1b) after as little as 10 min exposure. The same circuit controls this behaviour in the adult. To identify the contribution of DA neurons to regulation of pigmentation larvae were exposed to sulpiride, a DA D2 receptor antagonist, because co-expressed GABA and NPY can contribute to regulation of is behaviour. Sulpiride treatment. blocked changes in skin pigmentation response to altered illumination (FIG. 1c), demonstrating the predomdnant role of DA neurons in this pathway in Xenopus larvae. The number of TH VSC neurons was not affected by sulpiride.

Immunostaining for pro-opiomelanocortin (POMC) and NPY in combination with the nuclear marker 1,5-bis{[2-(dimthylamino) ethyl]amino}-4,8-dihydroxyanthracene-9,10-ddone (DRAQ5) revealed that the number of POMC melanotrope cells was also unchanged. D2 receptor activation was then analyzed in the eye to determine if this circuit is involved in the adaptation to background jilumination, because DA has complex effects on the retina. The blanching effect of a D2 receptor agonist, guinpirole hydrochloride, on dark-adapted larvae remaining in the dark persisted to the same extent after bilateral eye enucleation in the dark, suggesting that retinal D2 receptor activation plays little part in the reguation of this adaptation. The D1 receptor agonist and antagonist, SKF-38393 and SCH23390, had the opposite effect on this behaviour, darkening and lightening pigmentation, respectively. Other DA nuclei do not participate in this circuit. The pineal gland, a light sensor controlling circadian oscillation of melatonin that initiates slow changes in pigmentation, is not involved in rapid background adaptation. These results demonstrate participation of DA VSC neurons in the pathway controlling^{.} this camouflage behaviour.

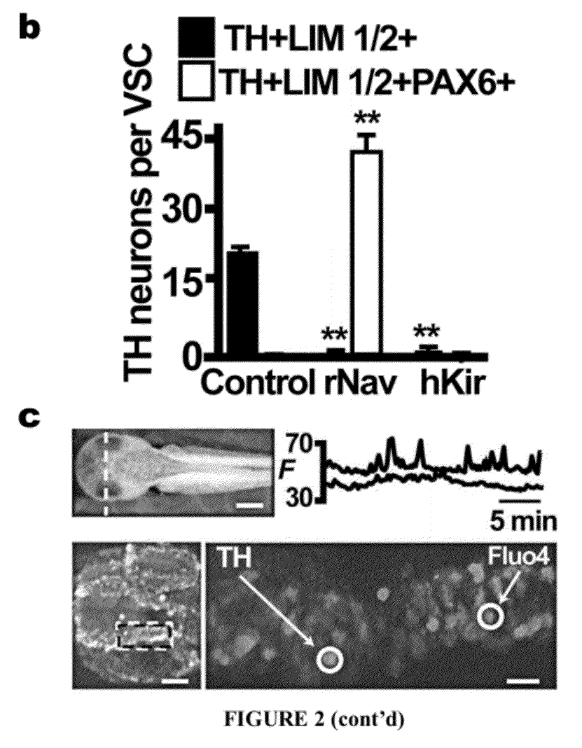

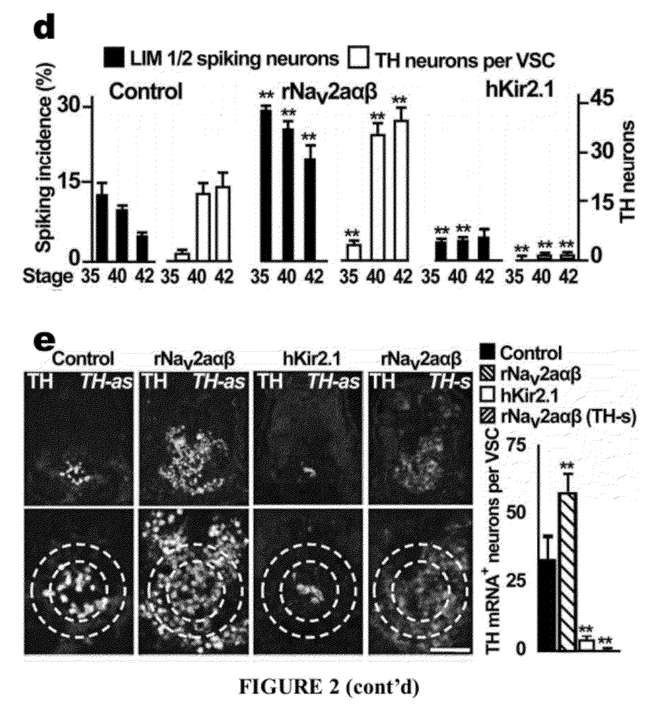

LIM1/2 and PAX6 transcription factor expression was analyzed during development to identify molecular signatures of TH VSC neurons. These neurons (FIG. 1d) were first detected at 2 days of development, and by 3 days they formed a nucleus with a core that ranged from 10 to 30 neurons depending on the illumination conditions in which the larvae were raised. VSC neurons were recognized by their expression of the LIM1/2 transcription factor and the absence of expression of PAX6 during the first 4 days of development (FIG. 2a, b, control).

To determine whether the temporal and spatial parameters of activity have a role in selective neuronal recruitment for neurotransmitter respecification, the effect of sustained and global changes of activity were compared throughout the central nervous system (CNS) with brief changes in circuit-specific activation. To test the general activity-dependence of DA expression, the effect of ion channel overexpression on the number of DA neurons was analyzed. Intracellular calcium was confocally imaged. with the Fluo-4AM indicator to determine whether neurons in the brain generate spontaneous calcium spikes. Neurons in the developing hypothalamus showed calcium spikes similar to those regulating transmitter specification in the posterior neural tube; fixing and immunostaining these preparations identified inactive neurons that express TH (FIG. 2c). These results suggested that, as in the spinal cord, calcium spikes precede the appearance of the transmitter, and raised the possibility that here too calcium spike. activity regulates transmitter expression.

To test this hypothesis inward rectifier potassium channels (human Kir2.1) Or voltage-gated sodium channels (rat Nav2aab) overexpressed at early developmental stages to suppress or enhance calcium spike generation. Increasing activity increased the number of TH neurons in and around the VSC, whereas suppressing activity decreased the number of TH neurons (FIG. 2a, b). Similar changes Were observed for the dorsolateral suprachiasmatic nucleus. Changes in the incidence. of calcium spiking (percentage of neurons spiking per h) during development Were inversely correlated with changes in TH expression, establishing a quantitative connection between the two (FIG. 2d). Increasing activity also induced expression of PAX6, but not LIM1/2, and newly TH neurons were recruited from the larger PAX6 and stable LIM1/2 pool of cells (FIG. 2a, b). In situ hybridization showed that increasing calcium spike. generation leads to widespread expression of X. laevis TH messenger RNA in cells not normally expressing it; decreasing calcium spike generation leads to a decrease in the number of cells expressing these transcripts (FIG. 2e). Thus, the changes in DA phenotype are regulated. transcriptionally. These findings indicate that spontaneous calcium spike activity controls the number of VSC DA neurons to regulate circuit function and skin pigmentation, because these neurons inhibit melanotrope cells, whereas global ion channel misexression has widespread effects that seem less specific.

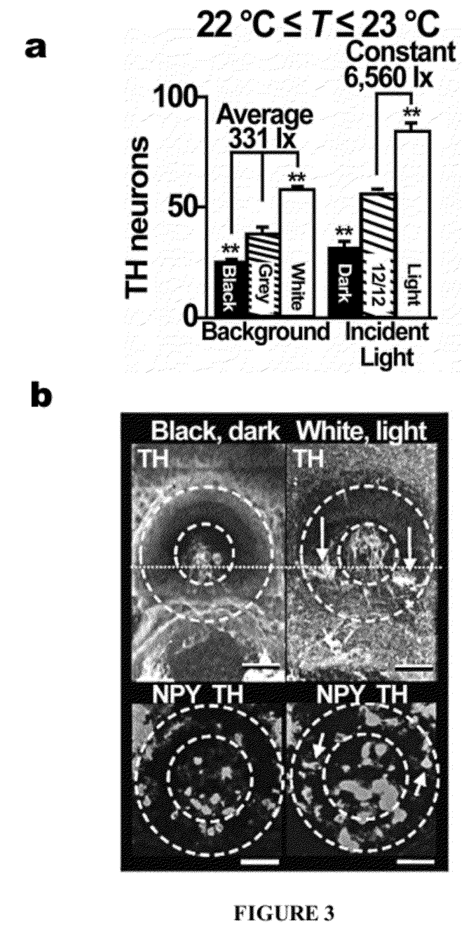

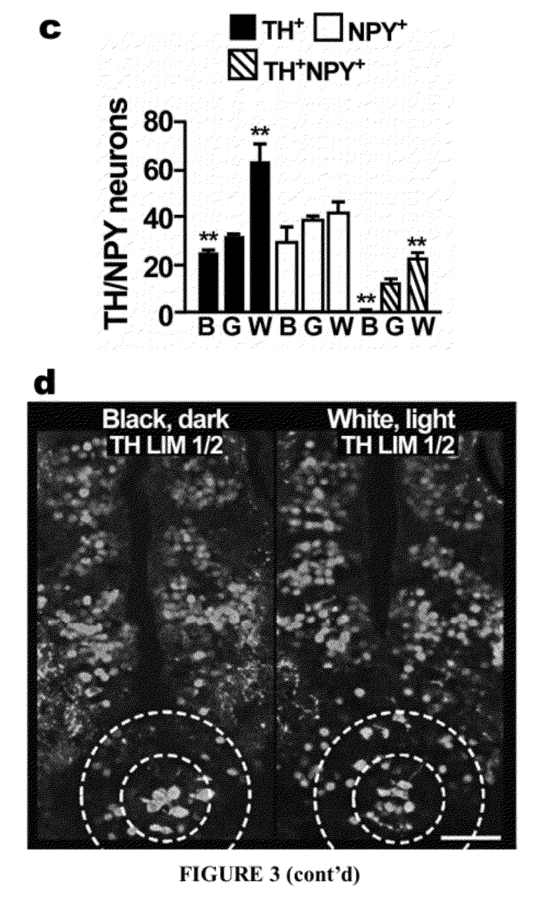

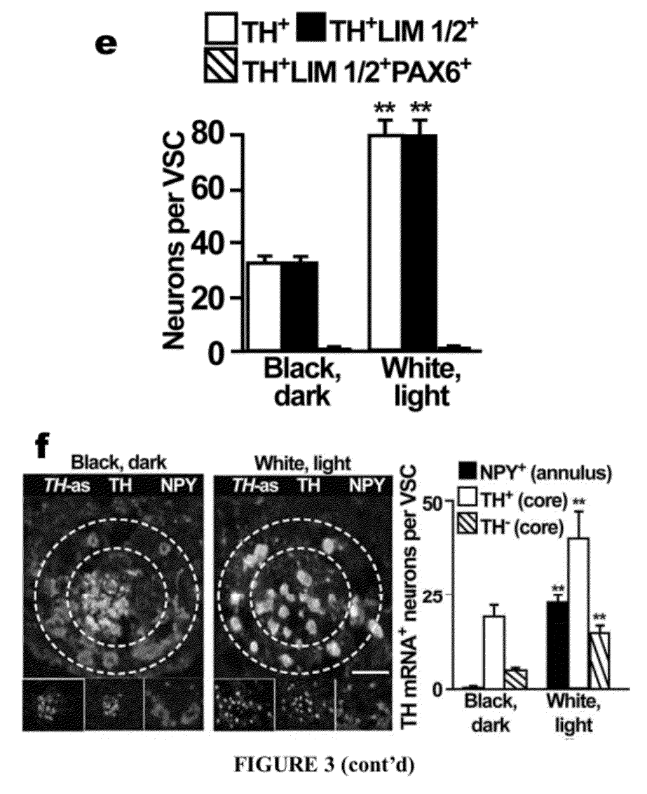

Light-dependence of dopamine expression. To determine whether newly dopaminergic neurons can be selectively recruited in specific circuits the effect of activating the retinohypothalamic projection was analyzed on neurotransmitter expression of VSC neurons. Raising animals in different levels of illumination and background changed the number of TH VSC neurons (FIG. 3a), and light on a white background was most effective in inducing an increase. Exposure of dark-raised animals to this illumination protocol for as little as 2 h led to the appearance of TH neurons in the annular region surrounding the core (FIG. 3b). By immunostaining neurons with antibodies to DA, the DA transporter (DAT) and vesicular monoamine transporter (VMAT2), the data demonstrates that all co-regulated with TH: all four markers were undetected in annular neurons of larvae kept in the dark and expressed in the NPY annular neurons of larvae that were exposed to 2 h light. During the respecification process these annular DA neurons are immunoreactive for FEY and for LIM1/2 but not for PAX6 (FIG. 3b-e). Thus, the more restricted physiological stimulus, in contrast to ubiquitous ion channel overexpression, recruits newly dopaminergic neurons from a restricted population. The absence of PAX6 induction may be due to the difference in the extent of altered activity. Increasing light exposure led to the appearance of TH mRNA. in annular NPY neurons.

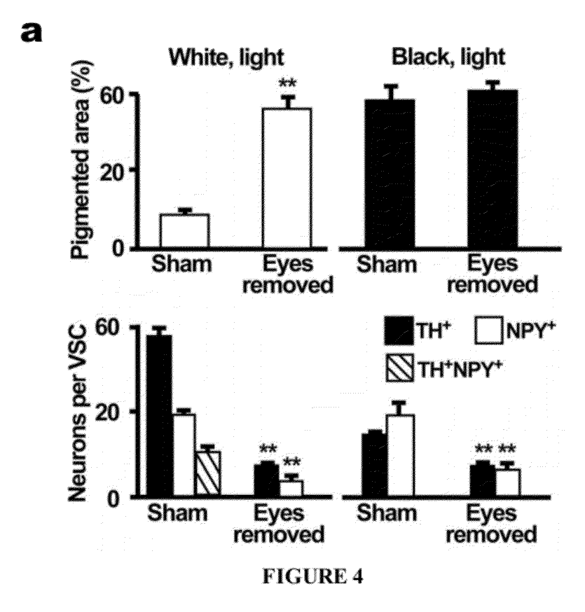

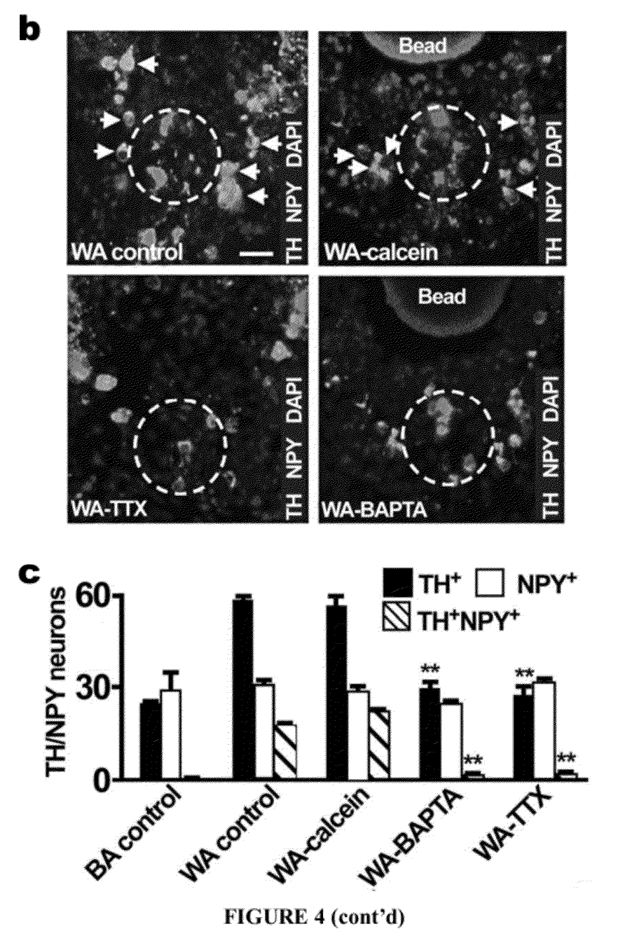

This transmitter respecification was reversible; decreasing light exposure reduced the extent of expression of TH mRNA in neurons in both. the annulus and the core of the VSC (FIG. 3f), and TH protein was no longer detected in 90% of TH annular neurons when larvae were kept in the dark for 2 h. Light-dependent acquisition of TH by NPY annular neurons in the VSC of dark-adapted larvae produces a new set of DA neurons that could regulate pigmentation, because NPY neurons are reported to innervate melanotrope cells in the adult and inhibit MSH production in a sustained manner. There was no change in the total number of 4,6-diamidino-2-phenylindole (DAPI)- or LIM1/2-stained nuclei, implying that neither cell proliferation nor migration account for the observed change in the total number of DA neurons. 5-bromodeoxyuridine (BrdU) labeling and TdT-mediated dUTP nick end labelling (TUNEL) assays did not reveal apoptosis or cell birth in these cells. The number of melanotrope cells remained constant, whereas the. number of POMC melanotrope cells increased after light exposure and decreased in the dark, associated with changes in intensity of POMC staining. Similar light-dependent accumulation of POMC in melanotrope cells occurs in white-background-adapted adult frogs. Suppressing activity eliminated DA expression elicited by light. Binocular eye enucleation that eliminates retinal input abolished both white. background adaptation and light-induced changes in the number of TH neurons (FIG. 4a).

A pharmacological approach to test the involvement of electrical activity and calcium signaling was then performed. 80-mm agarose beads loaded with calcein acetoxymethylester (calcein-AM) tracer were implanted in the roof of the hypothalamus above the VSC to evaluate the time course of drug delivery.

Twenty-four hours after implantation, the spread of tracer from the bead was localized within a radius of about 100 μm that included the VSC. Larvae implanted with beads loaded with the sodium channel blocker tetrodotoxin (TTX) or with the calcium. buffer 1,2-bis-(o-aminophenoxy)-ethane-N,N,N9,N9-tetraacetic acid, tetraacetoxymethyl ester (BAPTA-AM) (similar in molecular weight and diffusion profile to calcein-AM) showed no increase in number of either TH neurons or NPY/TH annular neurons in the VSC after 2 h illumination in white light following 24 h in the dark (FIG. 4b, c).

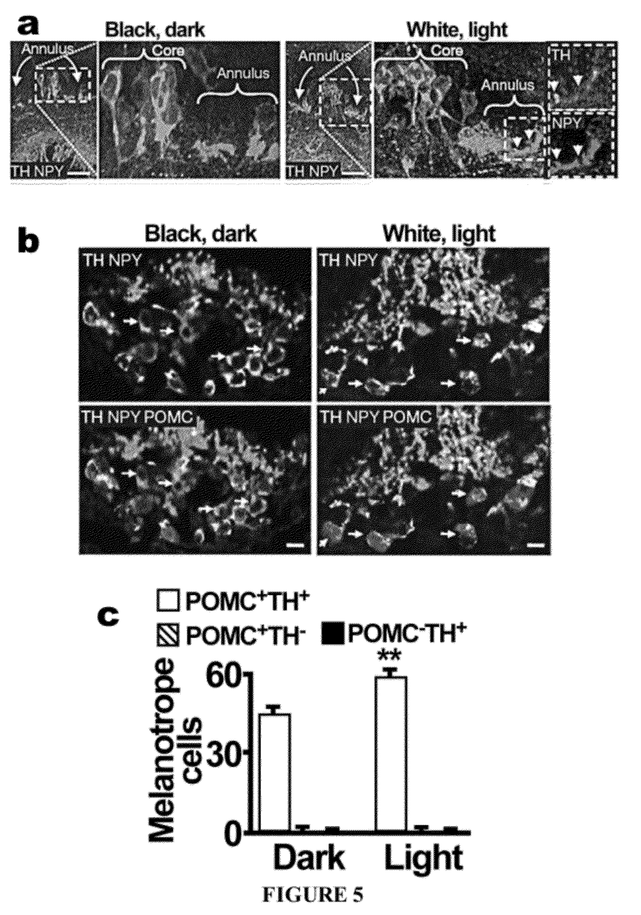

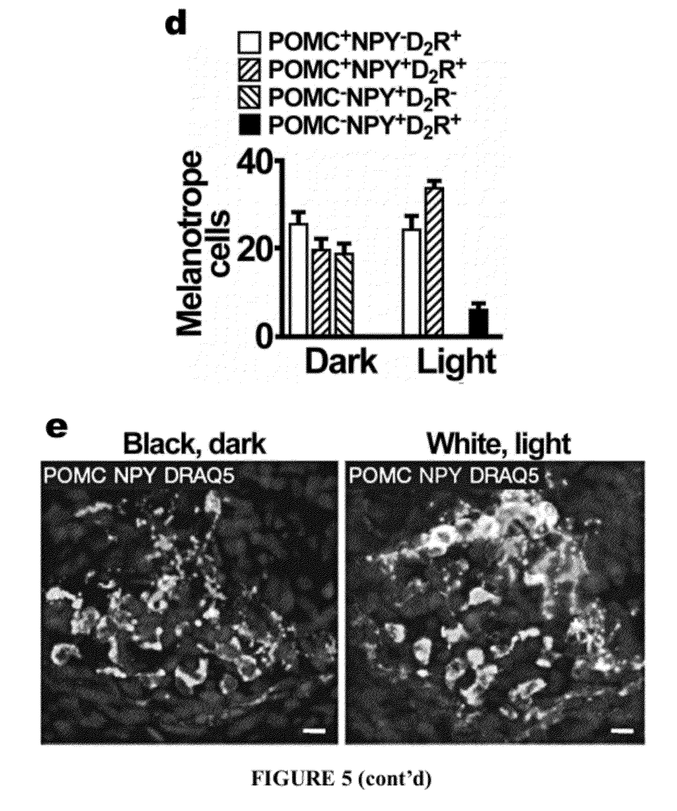

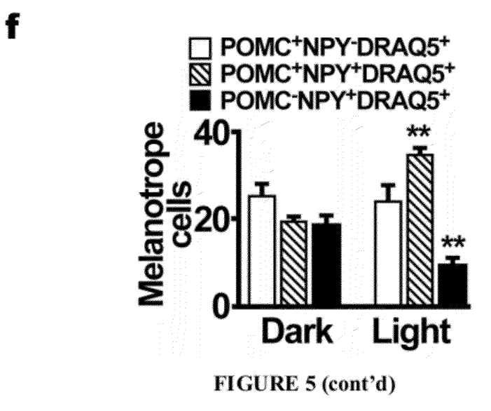

Function of light-induced dopamine neurons. To assess the behavioural potential of newly dopaminergic neurons a determination was made on whether they project to appropriate targets. After adaptation in the light for a minimum of 2 h, annular neurons, identified by LIM1/2 and NPY expression, co-expressed TH in their somata and axonal projections to the melanotrope cells (FIG. 5a). Localization of TH/NPY immunoreactivity adjacent to melanotrope cells, identified by staining for POMC, showed that NPY terminals are present in dark-adapted larvae and become TH/NPY in white-adapted larvae (FIG. 5b). POMC was not detected in a subpopulation of melanotrope cells that received only NPY terminals, but appeared after white adaptation when closely apposed nerve terminals became both NPY- and TH-positive (FIG. 5b, c). Moreover, D2 receptors were not observed at NPY terminals in dark-adapted larvae but were evident at TH/NPY terminals of white-adapted larvae (FIG. 5d).

These results suggest that the inhibitory output from the increased number of TH-expressing neurons in the VSC stimulates POMC storage in a larger number of melanotrope cells (FIG. 5e, f) and in turn reduces MSH release, which may account for accumulation of POMC in the melanotrope cells of white-background-adapted adult frogs.

To determine whether the larger population of DA neurons enhances the light sensitivity of changes in pigmentation, white-adaptation of larvae raised in the dark were compared to those raised in the light. These larvae were black-background-adapted for 30 min to achieve the same starting level of pigmentation before being tested.

Larvae raised in the light, which have more core TH neurons as well as TH/NPY annular neurons, white-adapted more rapidly and to a greater extent than larvae raised in the dark, whch have only core TH neurons. Prolonged white adaptation caused increases in the number of both core and annular TH neurons, with a slower time course than the initial changes in pigmentation. These results suggest that transmitter respecification provides a behavioural advantage. In contrast, dark adaptation of larvae with DA annular and core neurons placed on a dark background for 30 min occurred at the same rate as in larvae with only DA core neurons. This result is consistent with dark adaptation in adult frogs, which occurs in the absence of TH VSC inhibition of the melanotrope cells and by means of a different circuit involving the activity of serotonergic raphe neurons. These dynamics contribute to plasticity at the neuroendocrine-melanotrope interface.

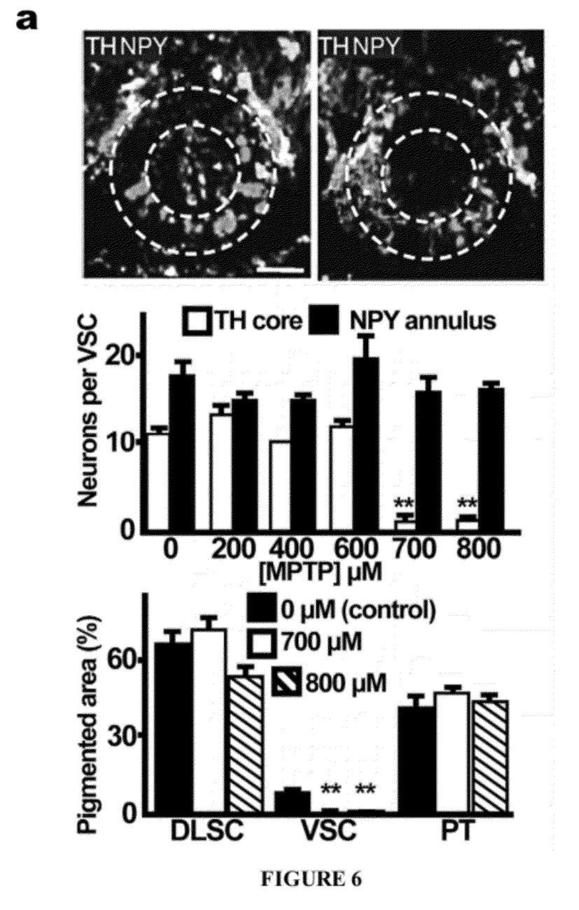

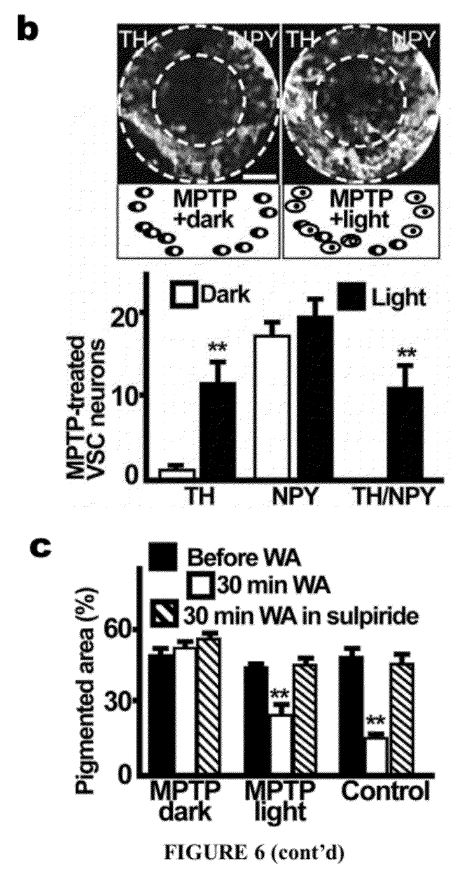

To investigate the independent function of these newly dopaminergic neurons the core DA neurons were selectively eliminated. By testing a range of concentrations of the dopaminergic toxin 1-methyl-4-phenyl-1,2,3,6-tetrahydropyridine (MPTP), the data shows that 700 μM MPTP for 8 h kills the core VSC neurons while sparing other DA nuclei (FIG. 6a). Because the number of TH neurons in the VSC remains constant for the first 40 min of light adaptation dark-raised, MPTP-treated larvae were evaluated after 30 min exposure to light, and this treatment abolished changes in pigmentation (FIG. 6b, c, left).

To determine the behavioural role of newly dopaminergic neurons, animals treated with MPTP and lacking VSC core neurons were exbosed to illumination in the absence of MPTP and the presence of deprenyl to block production of activated MPTP. This protocol again led to induction of TH/NPY annular neurons in the VSC (FIG. 6b), consistent with recruitment from the NPY population. Notably, these neurons alone drive changes in pigmentation in response to light. A high level of ambient illumination led to aggregation of melanin granules in melanocytes, and a low level of liuminatdon led to their dispersion, as in controls (FIG. 6c). These changes in pigmentation were blocked by exposure to sulpiride, identifying the contribution of DA neurons.

Imaging: Neural tubes dissected from three embryonic epochs and dissociated cell cultures prepared from neural-plate-stage embryos were loaded with 5 μM fluo-4 acetoxymethyl ester or 1 mM bisoxonol, and images were acquired at 0.2 Hz for 1-h periods with a BioRad MRC1024 laser confocal system. Spikes were stimulated at different frequencies in vitro by culturing neurons in 250 ml Ca²⁺-free saline medium by using a volume reducer and continuously superfusing them with this medium at 2.5 ml min⁻¹. The composition of saline was automatically switched for 15-20 s by computer-controlled solenoid valves (General Valve Corp.) to a solution containing 100 mM KCl and 2 mM Ca²⁺.

Molecular biology and pharmacology: hKir2.1, rNav2aab and rGluR2 were gifts from E. Marban, W. Catterall, and S. Heinemann. The genes were subcloned into a Bluescript vector and complementary DNA was transcribed with the mMessage mMachine (Ambion). Capped RNA (5-10 nl of a 0.01-0.1 mg ml⁻¹ RNA solution in 10% MMR, 6% Ficoll) was co-injected with a Cascade blue or Rhodamine red 30-kDa dextran (30 mg ml⁻¹) into one or both blastomeres at the two-cell stage with the use of a picospritzer (Picospritzer III; Parker Instrumentation). Control injections consisted of fluorescent dextran alone. Agarose beads (80 μm; BioRad) were loaded for 1 h with a solution containing 200 nM calcicludine (Calbiochem), 10 μM GVIAq-conotoxin, 10 μM flunarizine and 10 μm tetrodotoxin, or 1 mM veratridine, or 1% BSA (all from Sigma) before implantation. The effect of most of these agents on spike activity was tested at one-tenth of these concentrations; veratridine was tested at one-thousandth.

Immunocytochemistry: Embryos were fixed in 4% paraformaldehyde, 0.1% glutaraldehyde, phosphate-buffered saline (pH 7.4) for 2 h at 4° C., soaked in 30% sucrose for 2.5 h, and embedded in OCT compound (Tissue-Tek, Fisher Scientific). Frozen sections 10 μm in thickness were made over a 400-μm region of the neural tube starting about 400 μm posterior to the back of the eyes for control and channel-misexpression embryos. In bead-implanted embryos the bead was inserted between the neural tube and myotomes about 400 μm behind the eye primordium and sectioned from 100 μm anterior to 100 μm posterior to the bead. Cultures were fixed for 5-10 min with the same fixative. Slides and cultures were incubated in a blocking solution of 1% goat serum, fish gelatin or BSA for 0.5 h at 20° C., followed by incubation overnight at 4° C. with primary antibodies, and incubation for 2 h with fluorescently tagged secondary antibodies at 20° C. Immunoreactivity was examined on a Zeiss Photoscope with a 40× oil-immersion objective (or 20× and 40× water-immersion objectives for cultures) using a Xenon arc lamp attenuated by neutral density filters, and the appropriate excitation and emission filters for Cascade Blue, Alexa 488 and Alexa 594 fluorophores. Images were acquired and analyzed with Metamorph software (Universal Imaging Corp.). Each phenotype was scored in 20-30 consecutive sections or in cultures from at least five embryos. Immunoreactivities are presented in saturated color to clarify the distinction between positive and negative cells and to normalize image intensities. Differences from control were considered significant at p<0.05 (Student's t-test). Antibodies were from Affinity Bioreagents, Calbiochem, Chemicon and Sigma.

Electrophysiology: Cultured neurons and myocytes (myoballs) were observed with an upright compound microscope and a 40× water-immersion objective that allowed the positioning of electrodes with phase-contrast optics. Whole-cell recording was used to record SSCs. A Dell Dimension 4100 computer with Axon Instruments (PClamp 8.1) software and data interfaces was used for the acquisition and analysis of currents. Electrodes were pulled from borosilicate capillaries and had resistances of 3-5 MQ; they were filled with 100 mM KCl, 10 mM EGTA, 10 mM HEPES, pH 7.4. A perfusion system allowing rapid change of the bathing medium was used to achieve solution changes (2 ml min⁻¹). The receptor dependence of currents was determined by adding various drugs.

Another method examines changes at the cellular and molecular level, combining autoradiography of brain sections from PET experiments with immunocytochemistry for D2 receptors and tyrosine hydroxylase. Animals will be sacrificed after different light exposure regimens and the role of light-induced electrical activity in generating these changes by minipump infusion of glutamate receptor antagonists to block light-dependent activation of the retino-hypothalamic pathway during periods of altered light exposure is examined.

The methods will also examine behavior of adult rodents following sustained changes in ambient illumination. Many adult humans subjected to long nights at high latitudes during the fall and winter seasons suffer from seasonal affective disorder (SAD); treatment by phototherapy (exposure to light) is as effective as pharmacological enhancement of dopaminergic signaling in symptom remission during winter months. The mental status of adult rodents will be determined by the forced swim test (swim vs float), the open field test (for overall activity-control), reward-seeking (saccharin consumption), aggression (resident-intruder test) and anxiety (elevated plus-maze). Both laboratory rats (nocturnal) and Mongolian gerbils (diurnal) will be studied.

Neurotransmitter (NT) specification is regulated by calcium-dependent electrical activity in the developing Xenopus laevis embryo. This activity determines the numbers of neurons expressing classical neurotransmitters (glutamate, acetylcholine, GABA, and glycine) in the spinal cord and the numbers of neurons expressing monoamines in the brain. Electrical activity triggered by selective sensory circuit activation in the brain can also regulate the number of interneurons that express a specific NT, focusing on the dopaminergic VSC neurons following activation of the retino-hypothalamic projection. Regulation is achieved by co-expression of an additional transmitter by reserve pool neurons that already innervate the correct target.

To test whether circuit-specific recruitment of reserve pool neurons is a general phenomenon in the brain, the effect of the activation of another sensory modality, olfaction, was examined. Olfactory-mediated kin recognition in tadpoles is used to distinguish kin siblings from non-kin siblings via olfactory kinship cues, and 4-day old larvae display aversion behavior to water-borne non-kin odorants. Different classes of interneurons in the glomerular/periglomerular regions of the olfactory bulb were identified by tyrosine hydroxylase, GABA, NPY, and VGlut1,2 immunocytochemistry and further characterized by expression of transcription factors Pax6 and Lim1,2 and nuclear markers DAPI/DRAQ5.

Isolated stage 39 tadpoles were exposed to either kinship or non-kinship odorants for 24 hr and NT expression in olfactory bulb interneurons at stage 42 was compared to controls (exposed to blank saline for 24 hr). The results show that non-kin exposure leads to an increase (82±12%) in the number of GABAergic interneurons and to a simultaneous reduction (34±9%) of NPY co-expression within GABAergic cells of the periglomerular region, while the number of dopaminergic (TH+Pax6+) cells remains constant. Interestingly, exposure to kinship cues did not affect the number of GABAergic periglomerular interneurons, but caused a similar reduction (46±3%) in NPY expression. The data provide a novel clinical approach to selectively replenish NT expression in diseased brains via circuit activation by natural stimuli.

Following 48 hr exposure to either kinship or non-kinship odorants, stage 45 larvae were analyzed to assess NT expression in identified OB interneurons and tested for kin recognition behavior. The extent of dopamine/GABA coexpression changes dramatically following kin (sibling condition) or nonkin (non-sibling condition) exposure, compared to odorant deprivation (orphan condition). Local delivery of BAPTA or TTX prevents these changes in NT phenotype, demonstrating that they are activity-dependent. Doublecortin and BrdU labeling show that newly-respecified neurons are recruited neither from undifferentiated neurons nor from proliferating cells. Using a novel assay to test kin recognition in stage 45 free-swimming larvae we find that different extents of dopamine/GABA coexpression regulate the way in which the OB circuitry processes kin/nonkin odorants and determine whether the sign of the response to these cues involves attractive or repulsive kinship recognition behaviors. Altering the ratio of dopamine/GABA coexpression in granule and periglomerular interneurons reverses kinship recognition behavior: orphan-raised tadpoles demonstrate repulsive responses to siblings while nonkin-raised larvae display an attractive response to non-siblings.

In other experiments, adult male Long Evans rats were exposed to long and short day photoperiods and the number of TH-IR neurons in the hypothalamus was scored relative to controls. Three rats in one photoperiod chamber received 12:12 L:D illumination (12 hr in the light and 12 hr in the dark; controls) and 3 rats in another photoperiod chamber received either 19:5 L:D illumination (19 hr in the light and 5 hr in the dark; long photoperiod) or 5:19 L:D illumination (5 hr in the light and 19 hr in the dark; short photoperiod) for two weeks. TH-IR (dopaminergic) neurons were scored stereologically.

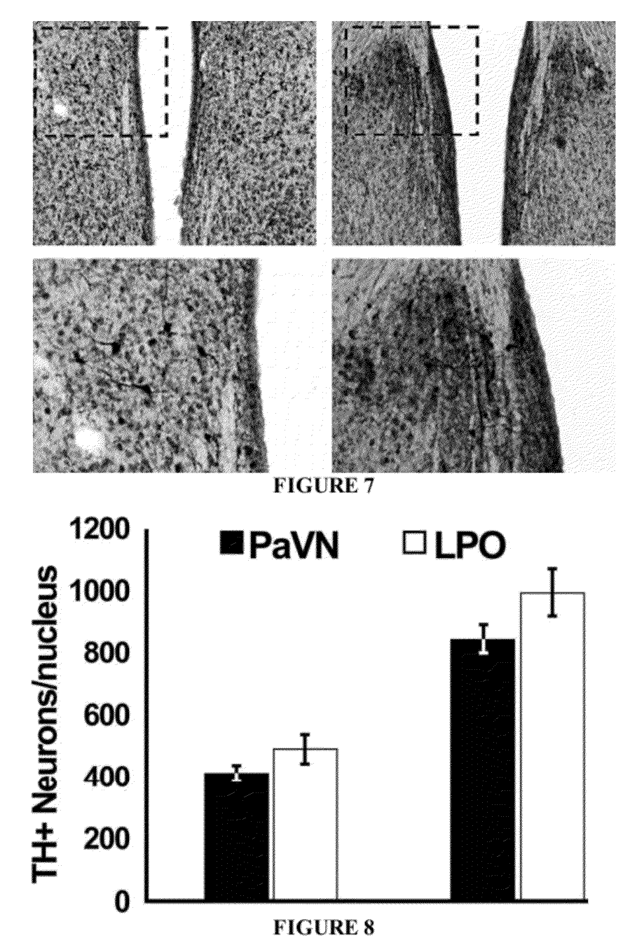

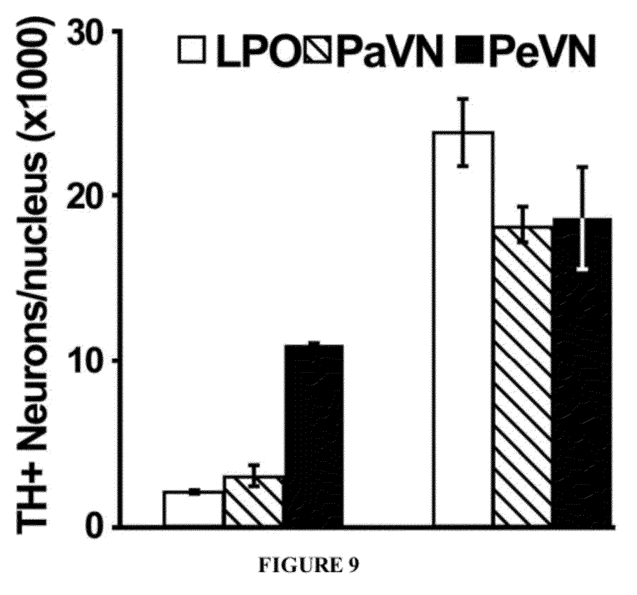

The number of TH-IR neurons was dramatically reduced in the paraventricular nucleus (PaVN) of rats exposed to the long day photoperiod relative to the brains of controls exposed to the 12:12 L:D photoperiod (FIG. 7). Decreases of 50% in the PaVN and lateral preoptic area (LPO) were evident (FIG. 8). These preliminary results suggested that changes in illumination change the numbers of neurons expressing dopamine in the adult rat hypothalamus. Accordingly, rats were exposed to manipulated photoperiods for a shorter time (1 week) and discovered that this stimulation was still effective (FIG. 9).

Such methods can be extended to humans. To extend the important animal findings to man, PET scans are carried out following exposure of normal human subjects to different light exposure, in parallel with the animal studies. These subjects will be exposed to bright white light and dim red light in separate sessions and receive PET scans following each exposure. Anatomical MRI will be obtained on each subject for gold-standard anatomical tracing of key brain structures.

The survey examines activity in the entire brain for activation by light. Based on a review of animal data, the hypothalamus, hippocampus, striatum and/or visual cortex are involved in circadian light effects. Human data have suggested prefrontal cortex and occipital cortex, but a systematic light exposure experiment using established light exposure methods has not been done. These areas will be examined in 24 normal subjects (selected as evening-active on standardized questionnaires). A group of 24 subjects will receive FDG-PET with absolute micromole quantification in one scan following bright white light and one following dim red light in random order between 5:30 and 9:00 AM. These scans will be carried out during the winter months to maximize the light effect.

The method also examines 18 FMT uptake to determine parallels with the animal studies. Subjects will be scanned following the same protocol and seasonal schedule described above.

The method also will examine dopamine receptors, again following the same protocol and seasonal method. Depending on whether the greatest light exposure differences are in the striatum or in the hypothalamus, amygdala or prefrontal regions, the study will be done in the winter with 18F-fallypride (for extrastriatal areas) or carry out 11C-raclopride (for the striatum) for seasonal exposure to light effects.

1. A method of modulating neurotransmitter specification in a neuron associated with the central nervous system, the method comprising contacting the neuron with a stimulatory factor that alters the pattern of Ca²⁺ spike activity of the neuron.

2. The method of claim 1, wherein the neuron is an embryonic neuron.

3. The method of claim 1, wherein the neuron is a mature neuron.

4. The method of claim 1, wherein the stimulatory factor is electrical, olfactory or light.

5. The method of claim 1, wherein the stimulatory factor is chemical.

6. The method of claim 1, wherein the neurotransmitter is acetylcholine, nitric oxide, histamine, norepineprhine, a bioactive amine, an amino acid or a neuropeptide.

7. The method of claim 6, wherein the bioactive amine is selected from the group consisting of dopamine, epinephrine, norepinephrine, serotonin.

8. The method of claim 6, wherein the amino acid is selected from the group consisting of glutamate, glycine and gamma-aminobutyric acid (GABA).

9. The method of claim 6, wherein the neuropeptide is selected from the group consisting of enkephalins, dynorphins and substance P.

10. The method of claim 1, wherein the modulation of neurotransmitter activity comprises altering neurotransmitter expression.

11. A method of treating or inhibiting a neurological or psychological disorder in a subject, the method comprising administering to the subject a stimulatory or inhibitory factor that alters the pattern of Ca²⁺ spike activity of neurons, wherein the treatment results in the modification of neurotransmitter activity produced by the neurons.

12. The method of claim 11, wherein the subject is a mammal.

13. The method of claim 12, wherein the mammal is a human.

14. The method of claim 11, wherein the psychological disorder is selected from the group consisting of addiction, substance abuse, autism, dyslexia, obsessive-compulsive disorder, generalized anxiety disorder, post-traumatic stress disorder, panic attacks, social phobia, major depression, bipolar disorder and schizophrenia.

15. (canceled)

16. A method of screening for factors that alter neurotransmitter expression in vivo comprising contacting cultures of neurons prepared from developing embryos, loaded with a calcium indicator, with a test stimulatory or inhibitory factor, wherein the neurons are exposed to different levels of the stimulatory or inhibitory factor and time-lapse imaging is used to assess changes in the firing pattern of calcium spikes produced by the neurons.

17. The method of claim 16 wherein the stimulatory or inhibitory factor is a chemical.

18. The method of claim 16 wherein the stimulant or inhibitory factor is electrical or light.

19. (canceled)

20. The method of claim 11, wherein the stimulant or inhibitory factor is a chemical.

21. The method of claim 11, wherein the stimulant or inhibitory factor is electrical or light.

22. The method of claim 21, wherein the electrical factor is applied in a manner mimicking mimicking natural patterns of activity occurring in the brain.

23. (canceled)

24. The method of claim 11, wherein the neurological disorder is selected from the group consisting of movement disorder, tardive dyskinesia, Huntington's disease, and Parkinson's disease.

25. (canceled)

26. The method of claim 1, wherein the neuron is an embryonic neuron.

27. The method of claim 1, wherein the stimulatory or inhibitory factor is electrical or light.

28. The method of claim 1, wherein the stimulatory or inhibitory factor is chemical.

29. The method of claim 28, wherein the stimulatory factor is a chemical such as veratridine.

30. The method of claim 28, wherein the inhibitory factor is selected from the group consisting of curare, tetrodotoxin, flunarizine, calcicludine, and omega-conotoxin.

31. The method of claim 1, wherein the neurotransmitter is acetylcholine, nitric oxide, histamine, norepineprhine, a bioactive amine, an amino acid or a neuropeptide.

32. The method of claim 31, wherein the bioactive amine is selected from the group consisting of dopamine, epinephrine, norepinephrine, and serotonin.

33. The method of claim 31, wherein the amino acid is selected from the group consisting of glutamate, glycine, and gamma-aminobutyric acid (GABA).

34. The method of claim 31, wherein the neuropeptide is selected from the group consisting of enkephalins, dynorphins, and substance P.

35. The method of claim 1, wherein the modulation of neurotransmitter activity comprises altering neurotransmitter expression.
